(19) United States
(12) Patent Application Publication
(10) Pub. No.: US 2022/0080135 A1
(43) Pub. Date: Mar. 17, 2022

(54) Unibody Small-Volume Nebulizer

(71) Applicant: Caddo Medical Technologies LLC, Dallas, TX (US)

(72) Inventor: Joseph Dee Faram, Dallas, TX (US)

(21) Appl. No.: 17/425,214

(22) PCT Filed: Jan. 24, 2020
(86) PCT No.: PCT/US20/14931

§ 371 Date: Jul. 22, 2021

Related U.S. Application Data
(60) Provisional application No. 62/798,586, filed on Jan. 30, 2019.

(51) Int. Cl. A61M 11/02; A61M 11/04

(52) U.S. Cl. CPC A61M 11/02; A61M 2207/00; A61M 2202/02; A61M 11/04

(57) ABSTRACT

The invention relates to a small-volume jet nebulizer having a unibody housing and an atomizer. The unibody housing includes input and output ports on a nebulizer body with an internal chamber that is widest at the output port. The housing also forms an internal gas inlet. The output port may be sized to fit standard patient interface ports. The nebulizer includes an atomizer that fits over the internal gas inlet. The atomizer includes a siphon, a jet and a baffle set a distance above the jet by arms connected to the siphon.

### CROSS-REFERENCE TO RELATED APPLICATIONS

This national stage application claims the benefit of priority from International Application No. PCT/US2020/014931 filed on Jan. 24, 2020, which claims the benefit of priority from U.S. Provisional Patent Application No. 62/798,586 filed on Jan. 30, 2019. Both applications are incorporated herein by reference.

### FIELD OF THE DISCLOSURE

The invention relates generally to the field of small-volume nebulizers.

### BACKGROUND

Medical nebulizers are used in hospitals, doctors' offices and home care environments for various reasons. Medical nebulizers are divided into two general categories: 1) large-volume, and 2) small-volume. Large-volume nebulizers are used, most often in hospital settings, to humidify gas, usually oxygen, to a patient. Large-volume nebulizers are utilized to add moisture to otherwise very dry gas by aerosolizing water, usually sterilized water with some mixture of saline in order to mimic the human body's salt content. Large-volume nebulizers often come pre-filled with various mixtures of sterile water and saline.

Small-volume nebulizers, also referred to as “hand-held nebulizers,” are used for delivering medication to the lungs. These devices are used for aerosolized medication therapy in both home and hospital settings. Although small-volume nebulizers are utilized in the delivery of a number of medications from analgesics to antibiotics, they are most often used to administer bronchodilators.

U.S. Pat. Nos. 9,566,397, 9,849,254 and 10,149,950 generally discuss small-volume nebulizers and pre-filled, small-volume nebulizers and are incorporated herein by reference.

Small-volume jet nebulizers featuring one input and one output are typically constructed with a top housing and a bottom housing, which attach to form a housing with a wide aerosolizing chamber, and a separate atomizer within the housing that corresponds to the internal shape of the bottom housing. The housing has an input port and an output port, which are both narrower than the central aerosolizing chamber in which the atomizer is located. This requires manufacturing at least three different components with three different molds.

More complex small-volume nebulizers may have alternative designs that allow for breath-enhanced or breath-actuated designs using one or more mechanical features and ambient air ports. Manufacturing requirements also increase in complexity with the additional features.

### SUMMARY

The present disclosure teaches a small-volume nebulizer with an atomizer within a unibody housing. The unibody housing may be manufactured from a single mold.

Some embodiments of the present disclosure provide a nebulizer body or housing with an input port and an output port. The output port's internal diameter is at least as large as the internal diameter of the remainder of the nebulizer body. In some embodiments, the internal diameter may be the same from the output port to the base of the nebulizer body. In other embodiments, the internal diameter may narrow from the output port down to the internal base of the nebulizer body. Because the internal diameter is largest at the top of the nebulizer's internal chamber, the nebulizer body may be formed on a single mold regardless of whether the internal diameter is the same throughout or narrows toward the bottom.

In some embodiments, the input port is the beginning of a gas inlet that extends into the nebulizer body opposite from the output port. The input port may be aligned with the output port.

An atomizer is configured to fit within the nebulizer body. In some embodiments, the internal base of the nebulizer body and the bottom of the atomizer have corresponding shapes that provide a limited intervening space, through which liquid may flow during operation. Embodiments of the atomizer may include a siphon, a jet and a baffle in-line with the jet.

The nebulizer includes the nebulizer body with the atomizer. The atomizer fits against the internal base of the nebulizer body with the jet in-line with the gas inlet.

The size of the nebulizer body may vary depending on operation. In some embodiments, the walls of the nebulizer body may extend above the baffle. In other embodiments, the walls may not reach the height of the baffle. In other embodiments, the walls may be approximately the same height as the baffle.

During operation, a nebulizer with medication is attached to a gas line on the input port and a patient interface on the output port. Gas enters the input port and passes through the gas inlet. As it exits the gas inlet, it passes through the intermediate space prior to entering the atomizer's jet. This gas flow causes medication to draw through the siphon, created by the intermediate space, and into the jet. Upon exiting the jet, the medication is impinged against the baffle, causing it to aerosolize within the nebulizer body. The user may then breathe the aerosolized medication through the patient interface.

In some embodiments, the nebulizer body may contain a unit-dose of medication sealed within it. A seal, such as a removable cap or pierceable seal, may cover the output port and input port to contain the medication. In some embodiments, the seal contacts the top of the atomizer.

Some embodiments include packaging to hold multiple nebulizers prior to use. Some packaging may include a tray fitted to hold the base of multiple nebulizers in place. The packaging may be configured for efficiently pre-filling medication and sealing the nebulizer prior to shipment.

Additional aspects, advantages and features are included in the following description of examples. The description should be understood in conjunction with the accompanying figures, wherein like numerals are used to describe the same feature throughout the figures. All patents, patent applications, articles and other publications referenced herein are hereby incorporated herein in their entirety for all purposes.

### A BRIEF DESCRIPTION OF THE DRAWINGS

Embodiments will now be described, by way of example only, with references to the accompanying drawings in which:

FIG. 1 is a perspective view of an embodiment of a unibody nebulizer with a patient interface and gas tube,

FIG. 2 is a cross-section view of an embodiment of a unibody nebulizer,

FIG. 3 is a cross-section view of another embodiment of a unibody nebulizer,

FIG. 4 is a cross-section view of an embodiment of a pair of unibody nebulizers in an embodiment of a packaging tray,

FIG. 5 is a perspective view of another embodiment of a unibody nebulizer attached to a patient interface,

FIG. 6 is an exploded view of the embodiment of a unibody nebulizer attached to a patient interface, and

FIG. 7 is a perspective view of an embodiment of an atomizer.

### DETAILED DESCRIPTION

While this invention may be embodied in many different forms, there will herein be described in detail preferred embodiments of the invention with the understanding that the present disclosure is to be considered as an exemplification of the principles of the invention and is not intended to limit the broad aspects of the invention to the embodiments illustrated. It will be understood that the invention may be embodied in other specific forms without departing from the spirit or central characteristics thereof. The present embodiments, therefore, are to be considered in all respects as illustrative and not restrictive, and the invention is not to be limited to the details given herein.

The elements of the small-volume nebulizer are described further in U.S. Pat. Nos. 9,566,397, 9,849,254 and 10,149,950, which may be referred to for further understanding of the present disclosure.

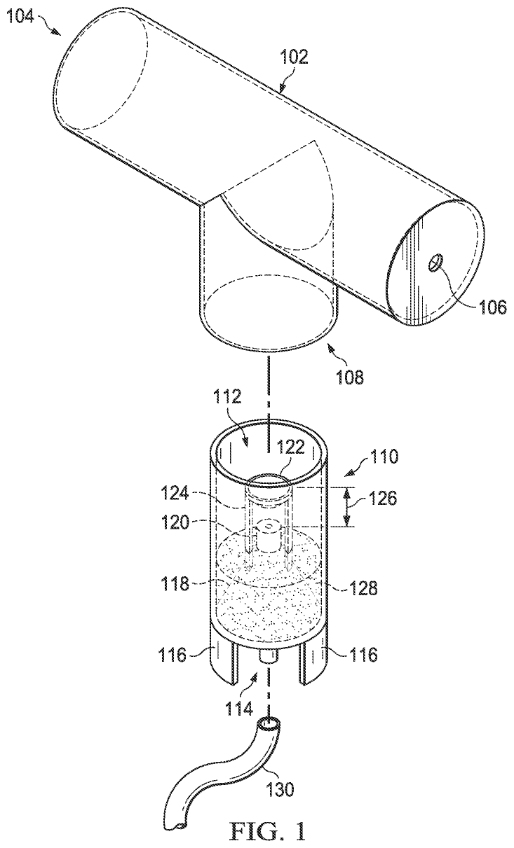

FIG. 1 illustrates an embodiment of a unibody nebulizer 110 with a T-connector 102 as a patient interface component and a portion of a gas input tube 130. The unibody nebulizer 110 includes a unibody nebulizer housing and an atomizer.

The T-connector 102 includes a patient opening 104 and a flow restrictor 106 on ends of the horizontal bar with the vertical port 108 located between the patient opening 104 and the flow restrictor 106.

The unibody nebulizer 110 includes an output port 112 and input port 114. The output port 112 is configured to fit into a patient interface component. For example, the output port 112 fits into the vertical port 108 of the T-connector 102. In some embodiments, the output port 112 may be configured to fit on the outside of the vertical port 108. The output port 112 may include an outer diameter that is configured to fit in a standard nebulizer port opening in a patient interface component. In other embodiments, the output port may be configured to fit with a specific patient interface, which may have a different shape or size than the standard nebulizer port opening.

In this embodiment, the input port 114 is located on the bottom of the nebulizer 110 opposite from the output port 112. A gas input tube 130 may be connected to the input port 114. The input port 114 may be a standard size input port or a specific size corresponding to the gas input tube 130. A flow of gas is provided through the gas input tube 130 into the input port 114 during operation.

Feet 116 are located on the nebulizer's 110 bottom edge. In this embodiment, the feet 116 extend downward a sufficient distance to allow the nebulizer 110 to stand on a surface with the input port 114 raised from the surface. In some embodiments, the feet 116 and input port 114 may extend the same distance allowing the input port 110 to further support the nebulizer 110. Some embodiments of a nebulizer 110 may not include feet 116.

The nebulizer 110 contains an atomizer which includes a siphon 118, jet 120 and a baffle 122. The baffle 122 is attached to the siphon 118 by arms 124 and is positioned in-line with the jet 120. The arms 124 hold the baffle 122 a set distance 126 above the jet's 120 output. This distance 126 may be based upon the desired aerosol characteristics, such as particle size, medication to air ratio or other characteristics.

As shown, the walls of the nebulizer 110 extend above the baffle 122. The internal diameter of the walls of nebulizer 110 is also the same from output port 112 to the bottom of nebulizer 110. In some embodiments, the internal diameter may narrow from the output port 112 to the base of the nebulizer 110.

At least during operation, the nebulizer 110 will contain medication 128 to aerosolize. When gas flows through the input port 114 into the nebulizer 110, the medication 128 will be drawn between the siphon 118 and the nebulizer's 110 base. The medication 128 will pass with the gas through the jet 120 and against the baffle 122. When the medication 128 impinges against the baffle 122, it disperses into the air in a plurality of droplets. This aerosolized medication 128 spreads into the internal space, or aerosol chamber, of the nebulizer 110. When a patient inhales on the patient interface component, the aerosolized medication 128 travels from the aerosol chamber through the T-connector 102 and into the patient's lungs.

The aerosol characteristics and air flow through the nebulizer may be tailored based upon the gas flowrate, the medication viscosity, the distance 126 between the jet 120 and the baffle 122, the nebulizer design, the patient interface design and other factors. Accordingly, a nebulizer may be designed for a specific medicine and desired aerosol particle size when implemented in a specific system.

In this embodiment, the nebulizer 110 produces aerosol continuously as long as the gas is flowing and medication 128 is in the nebulizer 110. In some embodiments, the nebulizer 110 may be attached to a patient interface that includes a breath-enhancement feature, such as a valve in the vertical port 108.

During manufacturing, the unibody housing may be created with a single mold. Similarly, the atomizer may be created with a single mold. Creating two single-mold components reduces manufacturing cost and time associated with existing nebulizers that require multiple components and additional molds. In addition, such nebulizers often require additional connections between elements to form the working components.

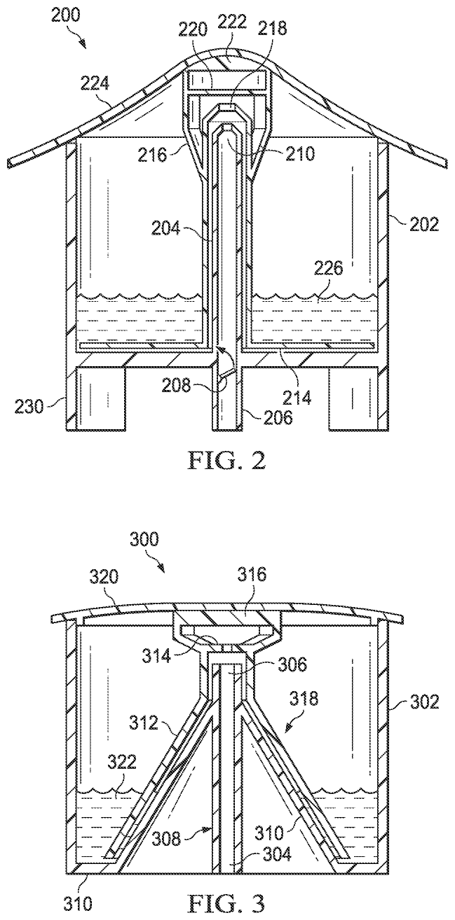

FIG. 2 illustrates a cross-section view of another unibody nebulizer 200. In this embodiment, the nebulizer 200 includes an exterior wall defining the nebulizer's 200 external body or housing ending in an output port 202 at the top of nebulizer 200. The output port 202 may be configured to connect to a patient interface's nebulizer port opening.

It also includes a gas inlet 204 extending from an input port 206 below the nebulizer's 200 base to a gas outlet 210 directed into the nebulizer 200. The input port 206 is configured to connect to a gas input. The input port 206 includes an inlet valve 208, which is a one-way valve configured to prevent medication 226 from escaping through the inlet port 206 while allowing gas to flow into the nebulizer 200.

An atomizer is within the nebulizer 200 and fits over the gas inlet 210. In this embodiment, the atomizer includes a siphon 214, arms 216, jet 218 and a baffle 220. The siphon 214 has a relatively flat base and a vertical tube that corresponds to the internal shape of the nebulizer's 200 base. This corresponding shape provides a limited space between the components, allowing the flow of gas from the gas outlet 210 to the jet 218 to cause a vacuum, pulling medication 226 through the siphon 214 and jet 218. When the medication 226 passes through the jet 218, it impinges against the baffle 220, creating an aerosol.

The baffle 220 is attached to the siphon 214 by arms 216 at a distance above the jet 218. In addition, a cover 222 is formed above the baffle 220.

In this embodiment, the output port 202 is covered with a seal 224. The seal 224 connects to the output port 202 and forms over the top of cover 222. As such, the seal 224 also acts to hold the atomizer in place within nebulizer 200 until the seal 224 is removed or otherwise opened.

This nebulizer 200 also includes feet 230 extending from the bottom of the housing. The feet 230 are designed to allow the nebulizer 200 to stand on a surface.

In preparation for an aerosol therapy treatment, a gas input tube is attached to the input port 206, the seal 224 is opened, and the output port 202 is connected to a patient interface's nebulizer port opening. During therapy, gas flows into the nebulizer 200 through the gas inlet 204, passing by the inlet valve 208 and out the gas inlet's 204 gas outlet 210. The gas passes from the gas outlet 210 through the jet 218 and against the baffle 220. As the gas passes between the gas outlet 210 and jet 218, the gas causes a suction, drawing medication 226 to an area between the siphon 214 and the base and gas inlet 204 of the nebulizer 200. The medication 226 passes through this area into the gas flow at the top of the gas inlet 204 and through the jet 218. This siphoned medication 226 impinges the baffle 220, causing the medication 226 to aerosolize.

In this embodiment, the aerosolized medication 226 may expand into a portion of the patient interface due to the reduced overall height of the nebulizer 200. In some embodiments, a patient interface component may include an aerosol chamber in which produced aerosol gathers until breathed in by a user.

Nebulizer 200 is able to aerosolize the medication 226 continuously as long as the gas is flowing and medication 226 is in the nebulizer 200. While the nebulizer 200 is not breath-actuated or breath-enhanced, it may be connected to a patient interface component or respiratory system that facilitates breath-actuated or breath-enhanced features.

FIG. 3 shows another embodiment of a nebulizer 300. Nebulizer 300 includes an outer wall 302 that forms an output port at the top of nebulizer 300. Nebulizer 300 further includes an inlet 308 having an input port 304 at the bottom nebulizer 300 and a gas outlet 306 at the top nebulizer 300, which is directed into the nebulizer 300.

In this embodiment, the base 310 of the nebulizer 300 has a flat outer portion adjacent to the outer wall 302 and a conical inner portion adjacent to the inlet 308 and angled upward toward the gas outlet 306. In this embodiment, the base's 310 flat outer portion acts as a support for nebulizer 300. To maintain a flat support, the input port 304 is not lower than the flat outer portion of the base 310.

The nebulizer 300 includes a unit-dose of medication 322 in the bottom of the nebulizer's 300 internal chamber, which is sealed at the top with seal 320. The seal 320 covers the output port at the top of the outer wall 302.

An atomizer 318 is also within the nebulizer's 300 internal chamber. The atomizer 318 includes a siphon 312, a jet 314 and a baffle 316 spaced a set distance from the jet 314. In this embodiment, a seal 320 contacts the top of baffle 316, thereby holding the atomizer 318 in place. The atomizer 318 is a conical shape corresponding to the base 310. The corresponding conical shapes leave a limited space for siphoning the medication 322 between the siphon 312 and base 310. The siphoned medication 322 enters into the gas stream at the jet 314.

To operate the nebulizer 300 as part of a respiratory treatment, a gas tube is attached to the input port 304. In addition, the seal 320 is opened, and a patient interface is attached to the output port.

During operation, gas flows into the input port 304 through the outlet 306 and into the jet 314. As the gas passes between the outlet 306 and jet 314, the gas flow causes medication 322 to pull through siphon 312 and into the gas stream, passing through the jet 314. Upon exiting the jet 314, the medication 322 impinges the baffle 316, causing the medication 322 to aerosolize in the nebulizer's 300 surrounding internal chamber. In some implementations, the aerosolized medication 322 may further expand into a patient interface's nebulizer port or aerosol holding chamber.

When a user inhales through the patient interface, medicated aerosol is drawn from an aerosol chamber, such as the nebulizer's 300 internal chamber, into the user's respiratory system.

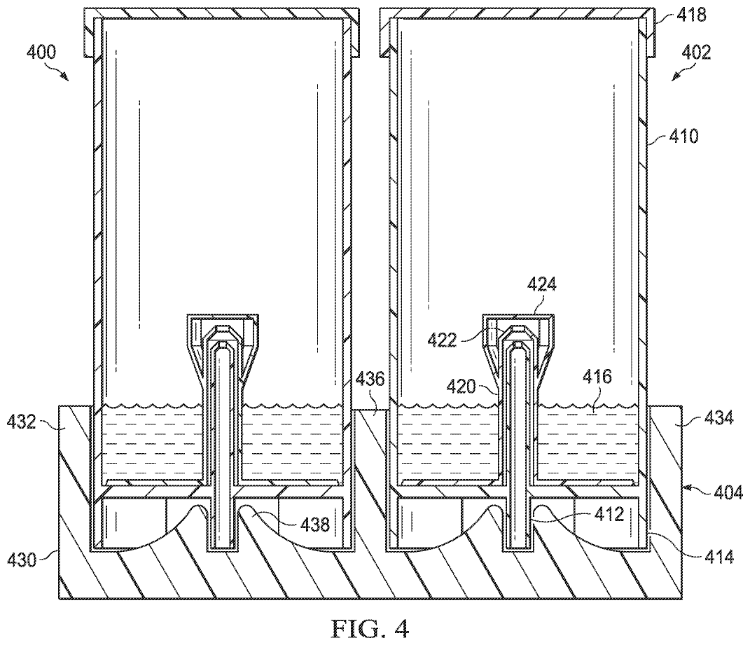

FIG. 4 depicts nebulizers 400 and 402 in a packaging tray 404. In this embodiment, nebulizers 400 and 402 are the same. In some embodiments, the nebulizers 400 and 402 may be different. For example, the nebulizers 400 and 402 may contain different medicine amounts for tapering off dosage. As another example, the nebulizers 400 and 402 may have different structures to modify aerosol particle size.

The nebulizers 400 and 402 include side wall 410 forming a cylindrical shape around the nebulizers' exterior. In some embodiments, the side wall 410 or walls may form an alternate shape, such as a polygonal tube. The top of the side wall 410 is an output port and the bottom forms feet 414. In this embodiment, the top of side wall 410 extends above the top of baffle 424.

In this embodiment, the base of the nebulizers 400 and 402 includes an inlet 412 having an input port at the bottom and an outlet in the nebulizers' 400 and 402 interior chambers. The output port at the top of side wall 410 is sealed with a removable cap 418 in this embodiment. In other embodiments, the seal is configured to open without removal. For example, it may be a pierceable seal that opens based on a puncture feature in a patient interface component.

Doses of medication 416 are sealed within the nebulizers 400 and 402 when packaged. Atomizers, which each include a siphon 420, a jet 422 and a baffle 424, are also included within the nebulizers 400 and 402. The baffle 424 is attached to the siphon 420 and positioned a set distance from the jet 422.

The packaging tray 404 is shown holding two nebulizers 400 and 402. In some embodiments, the packaging tray 404 may be configured to hold more or less nebulizers 400 and 402. For example, a packaging tray 404 may be configured to hold 14 nebulizers corresponding to a user's one-week supply for a twice-a-day therapy treatment.

Packaging tray 404 includes a base 430 with edge posts 432 and 434 and center post 436 extending upward to form two nebulizer slots, which are each able to hold a nebulizer. In some embodiments, these posts may include protrusions or extensions directed toward the nebulizers 400 and 402. Such protrusions may create a tighter fit around the nebulizers 400 and 402 and improve support of nebulizers 400 and 402 in the packaging tray 404.

In this embodiment, the packaging tray 404 also includes raised grips 438 configured to fit the inlet's 412 input port and provide an improved connection between the nebulizers 400 and 402 and the packaging tray 404. During the manufacturing or packaging process, each nebulizer 400 or 402 may be inserted into the packaging tray 404, causing raised grips 438 to engage the input port.

In some embodiments, a user may remove one nebulizer 400 or 402 from the packaging tray 404 to set up a therapy treatment. Once the nebulizer 400 or 402 is removed, the set up and therapy operation will be the same as the process described with other embodiments.

The packaging system, including nebulizers 400 and 402 and the packaging tray 404, may be designed to allow the user to open the removable seal or cap 418 and attach a patient interface component prior to removing the nebulizer 400 or 402 from the packaging tray 404. Once the nebulizer 400 or 402 attached to the patient interface component is removed, a gas tube is attached to the inlet's 412 input port. Thereafter, the therapy treatment operation continues as discussed with other embodiments.

In some embodiments, the packaging tray 404 is designed to hold the nebulizers 400 and 402 after they have been used. In such embodiments, the packaging system may be recycled after all medication has been used. In some embodiments, the packaging tray 404 may be designed to hold used nebulizers 400 and 402 in an upside down position with the output port engaged with the packaging to avoid confusion to the user as to which nebulizers 400 and 402 are used and those that are unused.

In some embodiments, the packaging tray 404 may be configured to fit a medicine filling machine or apparatus. For example, the packaging tray 404 may fit in a conveyor associated with a medicine filling machine. As the conveyor moves the packaging tray 404 with nebulizers 400 and 402 having an open output port, a series of filling nozzles dispenses a dose of medication 416 in each nebulizer 400 and 402. The filling machine may also include a device to place caps 418 on the nebulizers 400 and 402 in order to seal the medication within the nebulizer body.

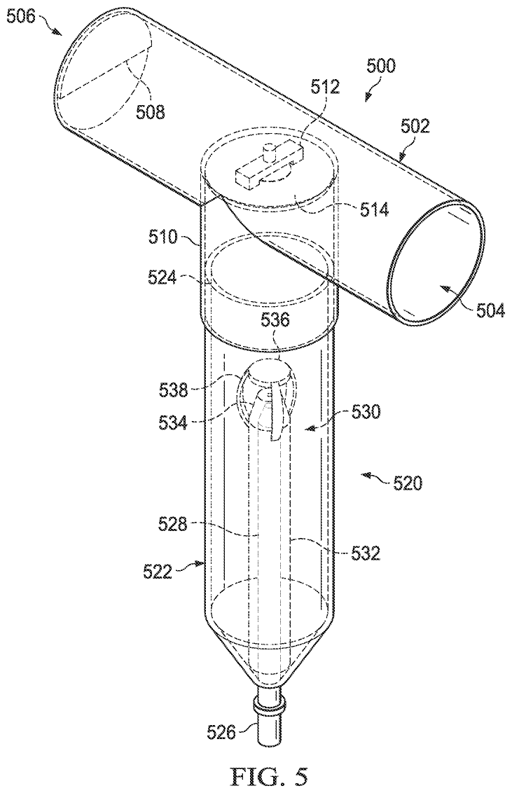

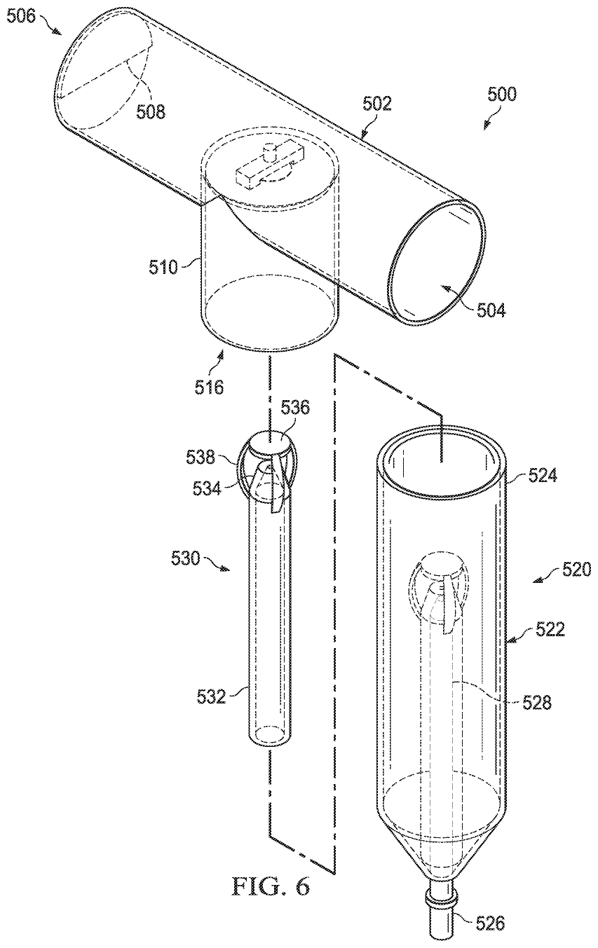

FIG. 5 provides an assembled view and FIG. 6 provides an exploded view of another embodiment of a nebulizer system 500. The nebulizer system 500 includes a patient interface 502 and a nebulizer 520. The patient interface 502 is shown as a T-piece, which includes a patient opening 504 on a first end of the horizontal bar and an ambient air opening 506 on the opposite, second end. In this embodiment, the ambient air opening 506 is restricted to around half the area of the second end with a wall 508 covering the upper half.

The patient interface 502 also includes a vertical port 510 between the patient opening 504 and ambient air opening 506. As shown in FIG. 5, the vertical port 510 includes a one-way valve within the patient interface to limit loss of aerosolized medication during operation. The one way valve includes the support bar 512 and valve gate 514. The incorporation of a one-way valve is disclosed further in U.S. Pat. No. 9,566,397, which is incorporated herein by reference.

The nebulizer 520 includes a nebulizer housing 522 or body having an output port 524 at the top of the nebulizer housing 522 and an input port 526 at the bottom of the nebulizer housing 522. The output port 524 is configured to fit into the patient interface's 502 vertical port 510. The input port 526 includes a gas opening that continues inside the open chamber within the nebulizer housing 522 as inlet 528. Prior to a therapy treatment, the input port 526 is connected to a source of gas.

In this embodiment, the base of the nebulizer 520 is angled downward from the wider section of the nebulizer housing 522 to the input port 526 forming a downward cone shape. During operation, this angled base can cause the medicine to gather closer to the inlet 528 throughout the therapy session, including when the last of the medicine is remaining. In some embodiments, the base may be flat or angled upward. Some bases may also have alternative appearances or designs for the tapered sections, including tapered concentric rings, curved or hemispherical shapes.

An atomizer 530 fits over the inlet 528. The atomizer 530 includes a siphon section 532 leading to jet 534. A baffle 536 is aligned above jet 534 at a set distance and held in place by arms 538. The atomizer 530 rests against the nebulizer's 520 base providing a limited space to allow medicine to flow under the atomizer's 530 edge. In some embodiments, the bottom of the atomizer 530 may include bumps or ridges to provide the space. Similarly, the atomizer 530 is configured to correspond with the inlet's 528 size, providing a limited space between the atomizer 530 and inlet 528 through which medicine may be siphoned during operation.

In some embodiments, the nebulizer housing 522 and atomizer 530 may include corresponding features to hold the atomizer 530 and nebulizer housing 522 together. For example, the atomizer 530 may have internal ribs within the siphon section 532 that grip the inlet 528. For another example, the interior surface of nebulizer housing 522 may include a rib or notch, which the atomizer's 530 arms 538 fit under or into, causing the atomizer 530 to stay in place within the nebulizer 520.

During operation, gas enters the nebulizer 520 through the input port 526 and inlet 528. The gas passes from the inlet 528 into the atomizer's 530 jet 534. As the gas flows from the inlet 528 to the jet 534, it pulls medicine through the space between the atomizer 530 and inlet 528 and into the gas flow entering the jet 534. The medicine exits the jet 534 and impinges against the baffle 536, which causes the medicine to aerosolize.

In some embodiments, the atomizer 530 is configured to provide aerosol for a medication at a specific particle size. The particle size may be based on the gas flow rate in conjunction with the size of the jet's 534 output orifice and the distance from the jet 534 to the baffle 536. As such, a series of atomizers 530 may be available having different jet 534 sizes and distances to the baffle 536. A person may select the proper atomizer 530, which corresponds to the gas flow rate and the desired particle size for the medication. In some embodiments, the atomizers 530 may be color-coded to indicate the particle size configuration.

When the user inhales, the valve gate 514 opens and aerosolized medication moves from the aerosolizing chamber within the nebulizer housing 522 and vertical port 510 to the user's lungs through the patient interface opening 504. When the user exhales, the valve gate 514 shuts, thereby holding aerosolized medication in the aerosolizing chamber. In addition, the limited ambient air opening 506 restricts the flow providing a non-medicated physiotherapy. In some embodiments, the patient interface 502 may include a variable flow restrictor. For example, a rotatable cap having a series of slots may be attached over the ambient air opening 506. The airflow through the ambient air opening 506 may be modified by rotating the cap to limit or increase the number of slots overlapping the ambient air opening 506.

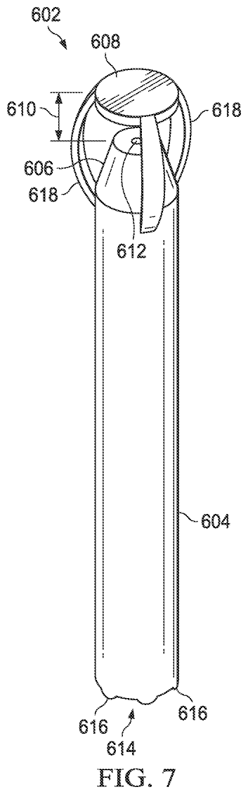

FIG. 7 illustrates an atomizer 602 for use within a unibody nebulizer. The atomizer 602 includes a siphon 604, a jet 606 and a baffle 608 spaced a distance 610 above the jet 606 by arms 618.

The siphon 604 extends from an opening 614 at the bottom of the siphon 604 to the jet 606 at the top of the siphon 604. The opening 614 and siphon 604 fit over a nebulizer's internal gas inlet. In this embodiment, the atomizer 602 includes a series of bumps 616, which are designed to create a limited space between a nebulizer's base and the siphon 604 to allow medication to flow under the siphon 604.

The jet 606 narrows to an outlet orifice 612 aligned directly below the baffle 608. The outlet orifice 612 may be aligned directly below a central area of the baffle 608 or off-center. When the atomizer 602 is on a nebulizer inlet, the outlet orifice 612 is above the gas flow from the inlet. The gas flow from the inlet and through the jet 606 causes medication to be pulled through the opening 614 and siphon 604 into the gas flow. The medicated gas flow exits the jet's 606 outer orifice 612 and impinges the baffle 608, creating the aerosolized medication.

Each of the nebulizer embodiments may be manufactured as two pieces—the nebulizer body and the atomizer. The output port of the nebulizer body is the same internal diameter as or larger than the base, allowing a mold configured to form the internal cavity to be removed from the output port. This mold can create a single-piece nebulizer body, including the base with an input port, a gas inlet, feet (if included in the embodiment) and side wall extending to the output port. Similarly, the atomizer may be molded as a single piece, which includes the siphon, jet and baffle attached to the siphon with one or more arms.

The invention being thus described and further described in the claims, it will be obvious that the same may be varied in many ways. Such variations are not to be regarded as a departure from the spirit and scope of the invention, and all such modifications as would be obvious to one skilled in the art are intended to be included within the scope of the apparatuses and methods described.

1. A jet nebulizer comprising:
a unibody housing comprising a base, a side wall and an inlet, wherein the side wall has an inner surface and an outer surface and forms an interior chamber that extends from the base at a bottom of the interior chamber to an output port at a top of the interior chamber, wherein the unibody housing has an internal diameter between opposite points on the inner surface, wherein the internal diameter is largest at the top of the interior chamber, and wherein the inlet comprises an internal portion extending within the interior chamber from the base toward the top and an external input port extending below the base; and
an atomizer within the interior chamber, the atomizer comprising a siphon, a jet and a baffle attached by arms a set distance directly above the jet, wherein the baffle attaches to a top of the arms and the atomizer corresponds to the base's shape and fits over the inlet and a portion of the base;
wherein the output port is sized to fit a nebulizer port in a patient interface, which is distinct from the jet nebulizer, and the input port is configured to attach to a gas input tube.

2. The jet nebulizer of claim 1, wherein the output port is opposite from the input port.

3. The jet nebulizer of claim 1, comprising a first seal over the output port and a second seal over the input port and a unit-dose of medication sealed within the unibody housing by the first seal and the second seal.

4. The jet nebulizer of claim 3, wherein the first seal contacts a top surface of the atomizer.

5. The jet nebulizer of claim 1, wherein the unibody housing comprises feet on the bottom.

6. The jet nebulizer of claim 1, wherein the base and the atomizer are conical in an upward orientation.

7. The jet nebulizer of claim 1, wherein the nebulizer is configured to continuously aerosolize medicine when gas is moving through the inlet.

8. The jet nebulizer of claim 1, wherein the base is a downward cone shape, and the atomizer fits into the downward cone shape.

9. The jet nebulizer of claim 8, wherein the atomizer includes a bump at the bottom, wherein the bump raises the atomizer off the base a sufficient distance to allow medication to move into the siphon.

10. The jet nebulizer of claim 1, wherein the atomizer extends above a top edge of the output port of the unibody housing.

11. A method of manufacturing a jet nebulizer comprising the steps of:
molding a unibody housing comprising a base, a side wall and an inlet from a single mold, wherein the side wall has an inner surface and an outer surface and forms an interior chamber, which extends from the base at a bottom of the interior chamber to an output port at a top of the interior chamber, wherein the unibody housing has an internal diameter between opposite points on the inner surface, wherein the interior chamber is largest at the top of the interior chamber, and wherein the inlet comprises an internal portion extending within the nebulizer housing from the base toward the top and an external input port extending below the base;
molding an atomizer comprising a siphon, a jet and a baffle attached by arms a set distance above the jet, wherein the atomizer corresponds to the base's shape;
placing the atomizer in the unibody housing, wherein the atomizer fits over the inlet and a portion of the base; and
wherein the output port is sized to fit a nebulizer port in a patient interface component, which is distinct from the jet nebulizer, and the input port is configured to attach to a gas input tube, and
wherein aerosol is able to exit the jet nebulizer across the output port during operation of the jet nebulizer with the patient interface component.

12. The method of manufacturing a jet nebulizer from claim 11 further comprising the steps of:
filling the unibody housing with a unit-dose of medication; and
sealing the unibody housing with a first seal over the output port and a second seal over the external input port.

13. The method of manufacturing a jet nebulizer from claim 11 further comprising the steps of:
sealing the external input port with a second seal;
placing the unibody housing with the atomizer in a packing tray configured to hold a plurality of unibody housings;
filling the unibody housing with a unit-dose of medication, while filling the plurality of unibody housings in the packing tray;
sealing the unibody housing with a first seal over the output port, while sealing the plurality of unibody housings.

14. A jet nebulizer medication delivery assembly, comprising:
a unibody housing comprising a base, a side wall and an inlet, wherein the side wall has an inner surface and an outer surface and forms an interior chamber that extends from the base at a bottom of the interior chamber to an output port at a top of the interior chamber, wherein the unibody housing has an internal diameter between opposite points on the inner surface, wherein the internal diameter is largest at the top of the interior chamber, and wherein the inlet comprises an internal portion extending within the interior chamber from the base toward the top and an external input port extending below the base;
an atomizer within the interior chamber, the atomizer comprising a siphon, a jet and a baffle attached by arms a set distance above the jet, wherein the atomizer corresponds to the base's shape and fits over the inlet and a portion of the base, and wherein the atomizer rests against the base; and
a patient interface having a nebulizer port, wherein the nebulizer port attaches to the output port of the unibody housing, and wherein the output port is sized to fit directly into the nebulizer port;
wherein the input port is configured to attach to a gas input tube.

15. The jet nebulizer medication delivery assembly of claim 14, wherein the output port is opposite from the input port.

16. The jet nebulizer medication delivery assembly of claim 14, wherein the patient interface comprises an aerosol chamber.

17. The jet nebulizer medication delivery assembly of claim 14, wherein the patient interface provides breath-actuated aerosol delivery.

18. The jet nebulizer medication delivery assembly of claim 17, wherein the patient interface comprises a breath-actuated valve.

19. The jet nebulizer medication delivery assembly of claim 17, wherein the atomizer extends above a top edge of the output port of the unibody housing.

20. The jet nebulizer medication delivery assembly of claim 14, wherein the output port's diameter is substantially equal to the base's diameter.
